(19) United States
(12) Patent Application Publication
(10) Pub. No.: US 2024/0325388 A1
(43) Pub. Date: Oct. 3, 2024

(54) DOSAGE FORMS FOR TYK2 INHIBITORS COMPRISING SWELLABLE CORES

(71) Applicant: Bristol-Myers Squibb Company, Princeton, NJ (US)

(72) Inventors: Umesh Kestur, North Brunswick, NJ (US); Sherif Ibrahim Farag Badawy, Dayton, NJ (US); Dory Koehler-King, Bend, OR (US); Craig Allen Sather, Bend, OR (US); Kyle Kyburz, Montrose, CO (US)

(21) Appl. No.: 18/026,704

(22) PCT Filed: Sep. 17, 2021
(86) PCT No.: PCT/US2021/050928

§ 371 Date: Mar. 16, 2023

Related U.S. Application Data
(60) Provisional application No. 63/080,030, filed on Sep. 18, 2020.

(51) Int. Cl. A61K 31/501; A61K 9/20

(52) U.S. Cl. CPC A61K 31/501; A61K 9/2086

(57) ABSTRACT

A swellable core dosage form comprises a dispersion of amorphous 6-(cyclopropancamido)-4-((2-methoxy-3-(1-methyl-1H-1,2,4-triazol-3-yl)phenyl)amino)-N-(methyl-d3)pyridazine-3-carboxamide.

### FIELD OF THE INVENTION

Dispersions of amorphous 6-(cyclopropaneamido)-4-((2-methoxy-3-(1-methyl-1H-1,2,4-triazol-3-yl)phenyl)amino)-N-(methyl-d₃)pyridazine-3-carboxamide (BMS-986165) described herein are used in controlled release dosage forms comprising swellable cores. The dosage forms may be administered to patients for the treatment of auto-immune and auto-inflammatory diseases such as an inflammatory bowel disease (IBD) and psoriasis.

### BACKGROUND OF THE INVENTION

Tyrosine kinase 2 (Tyk2) is a member of the Janus kinase (JAK) family of nonreceptor tyrosine kinases and has been shown to be critical in regulating the signal transduction cascade downstream of receptors for IL-12, IL-23, and type I interferons in both mice (Ishizaki, M. et al., “Involvement of tyrosine kinase-2 in both the IL-12/Th1 and IL-23/Th17 axes in vivo,” J. Immunol., 187:181-189 (2011); Prchal-Murphy, M. et al., “TYK2 kinase activity is required for functional type I interferon responses in vivo,” PLOS One, 7:e39141 (2012)) and humans (Minegishi, Y. et al., “Human tyrosine kinase 2 deficiency reveals its requisite roles in multiple cytokine signals involved in innate and acquired immunity,” Immunity, 25:745-755 (2006)). Tyk2 mediates the receptor-induced phosphorylation of members of the STAT family of transcription factors, an essential signal that leads to the dimerization of STAT proteins and the transcription of STAT-dependent pro-inflammatory genes. Tyk2-deficient mice are resistant to experimental models of colitis, psoriasis, and multiple sclerosis, demonstrating the importance of Tyk2-mediated signaling in autoimmunity and related disorders (Ishizaki, M. et al., “Involvement of tyrosine kinase-2 in both the IL-12/Th1 and IL-23/Th17 axes in vivo,” J. Immunol., 187:181-189 (2011); Oyamada, A. et al., “Tyrosine kinase 2 plays critical roles in the pathogenic CD4 T cell responses for the development of experimental autoimmune encephalomyelitis,” J. Immunol., 183:7539-7546 (2009)).

In humans, individuals expressing an inactive variant of Tyk2 are protected from multiple sclerosis and possibly other autoimmune disorders (Couturier, N. et al., “Tyrosine kinase 2 variant influences T lymphocyte polarization and multiple sclerosis susceptibility,” Brain, 134:693-703 (2011)). Genome-wide association studies have shown other variants of Tyk2 to be associated with autoimmune disorders such as Crohn's disease, psoriasis, systemic lupus erythematosus, and rheumatoid arthritis, further demonstrating the importance of Tyk2 in autoimmunity (Ellinghaus, D. et al., “Combined Analysis of Genome-wide Association Studies for Crohn Disease and Psoriasis Identifies Seven Shared Susceptibility Loci,” Am. J. Hum. Genet., 90:636-647 (2012); Graham, D. et al., “Association of polymorphisms across the tyrosine kinase gene, TYK2 in UK SLE families,” Rheumatology (Oxford), 46:927-930 (2007); Eyre, S. et al., “High-density genetic mapping identifies new susceptibility loci for rheumatoid arthritis,” Nat. Genet., 44:1336-1340 (2012)).

BMS-986165 refers to a compound of the following Formula (I)

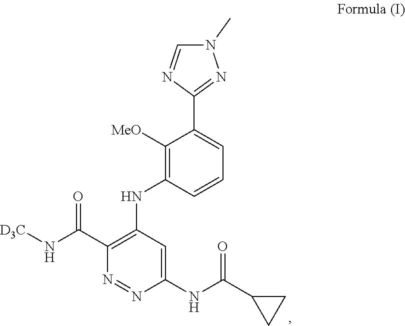 [SMILES: [2H]C([2H])([2H])NC(=O)c1nnc(NC(=O)C2CC2)cc1Nc1cccc(-c2ncn(C)n2)c1OC]

which is 6-(cyclopropaneamido)-4-((2-methoxy-3-(1-methyl-1H-1,2,4-triazol-3-yl)phenyl)amino)-N-(methyl-d₃)pyridazine-3-carboxamide. BMS-986165, which is under investigation for the treatment of auto-immune and auto-inflammatory diseases such as psoriasis, psoriatic arthritis, lupus, lupus nephritis, Sjögren's syndrome, inflammatory bowel diseases (including ulcerative colitis and Crohn's disease), and ankylosing spondylitis, is a highly selective inhibitor of Tyk2-mediated signal transduction. It selectively binds to the Tyk2 pseudokinase (JH2) domain and blocks receptor-mediated Tyk2 activation by stabilizing the regulatory JH2 domain.

BMS-986165 and other amide-substituted heterocyclic compounds useful as modulators of IL-12, IL-23, and/or IFNα responses, methods of making the same, and methods of using the same are disclosed in U.S. Pat. No. 9,505,748 B2, the contents of which are hereby incorporated by reference in their entirety herein. Other methods of synthesizing BMS-986165 are disclosed in U.S. Provisional Patent Application No. 62/478,789 and PCT/US2018/025100 (published as WO 2018/183649), the contents of each of which are hereby incorporated by reference in their entirety herein.

Formulations and dosage forms with swellable cores are described in U.S. Pat. Nos. 6,706,283 and 9,028,870, for example.

### DESCRIPTION OF THE INVENTION

The present invention provides methods of treating auto-immune and auto-inflammatory diseases in a patient, comprising: orally administering once daily to the patient a swellable core dosage form of 6-(cyclopropaneamido)-4-((2-methoxy-3-(1-methyl-1H-1,2,4-triazol-3-yl)phenyl)amino)-N-(methyl-d₃)pyridazine-3-carboxamide (BMS-986165) comprising a spray-dried dispersion of amorphous BMS-986165 in a polymer matrix. The auto-immune or auto-inflammatory disease may be, for example, an inflammatory bowel disease (such as ulcerative colitis or Crohn's disease) or psoriasis (such as plaque psoriasis). The dosage form is preferably a bi-layer tablet.

The present invention also provides methods of treating an inflammatory bowel disease in a patient, comprising: orally administering once daily to a patient a swellable core dosage form of 6-(cyclopropaneamido)-4-((2-methoxy-3-(1-methyl-1H-1,2,4-triazol-3-yl)phenyl)amino)-N-(methyl-d3)pyridazine-3-carboxamide (BMS-986165) comprising a spray-dried dispersion of amorphous BMS-986165 in a polymer matrix. The inflammatory bowel disease may be ulcerative colitis or Crohn's disease. The dosage form is preferably a bi-layer tablet.

The present invention further provides methods of treating psoriasis in a patient, comprising: orally administering once daily to a patient a swellable core dosage form of 6-(cyclopropaneamido)-4-((2-methoxy-3-(1-methyl-1H-1,2,4-triazol-3-yl)phenyl)amino)-N-(methyl-d3)pyridazine-3-carboxamide (BMS-986165) comprising a spray-dried dispersion of amorphous BMS-986165 in a polymer matrix. The psoriasis may be plaque psoriasis. The dosage form is preferably a bi-layer tablet.

The following examples serve only to illustrate the invention and its practice. The examples are not to be construed as limitations on the scope or spirit of the invention.

### EXAMPLES

### Swellable Core Formulation Comprising BMS-986165 SDD

BMS-986165-01 SDD (15% BMS 986165-01: 85% HPMCAS) was used in a swellable core formulation and dosage form. “BMS-986165-01” in this Example and throughout the present disclosure refers specifically to 6-(cyclopropaneamido)-4-((2-methoxy-3-(1-methyl-1H-1,2,4-triazol-3-yl)phenyl)amino)-N-(methyl-d₃)pyridazine-3-carboxamide in free base form. HPMCAS is hydroxypropyl methylcellulose acetate succinate (also referred to as hypromellose acetate succinate).

In this swellable core dosage form embodiment, the dosage form is a bilayer tablet comprising a drug layer and a sweller layer; each layer comprises an osmogen. The two layers make up the core, and the core is coated with a semipermeable coating. The drug is released through a laser-drilled hole on the drug-layer side of the bilayer. The semipermeable coating comprises a water insoluble polymer. Tables A1-A3 provide compositions for swellable core formulations. In addition, crystallization inhibitors may be included in the swellable core formulation, to prevent or reduce crystallization of BMS-986165.

The drug release rate of the swellable core formulation can be fine-tuned by varying the core composition, the coating composition, and/or the coating amount. For example, a swellable core tablet, dosed once-a-day, can achieve a drug release profile that is similar to the drug release profile achieved by twice-a-day dosing with an immediate-release tablet.

TABLE A-1
Swellable core formulation composition and ranges studied

| Components of drug layer | Function | Range (%) |
|---|---|---|
| BMS-986165-01 SDD (15% BMS | Active | 22-33 |
| 986165-01:85% HPMCAS) | | |
| Hydroxypropyl Methylcellulose | Sustaining polymer | 0-15 |
| Acetate Succinate (HPMCAS) | | |
| PolyOx (Molecular weight | Entraining polymer | 42-57 |
| 200,000-5,000,000) | | |
| Sodium chloride | Osmogen | 5-15 |
| Silicon dioxide | Glidant | 0.5-1 |
| Magnesium Stearate | Lubricant | 0.5-1 |
| Components of sweller layer | | |
| Microcrystalline cellulose | Filler | 20-30 |
| PolyOx (Molecular weight | Entraining polymer | 60-70 |
| 5,000,000) | | |
| Sodium chloride | Osmogen | 5-15 |
| Iron oxide | Colorant | 0.2 |
| Magnesium Stearate | Lubricant | 0.5-1 |
| Coating composition and amount | | |
| Cellulose Acetate | Film forming polymer | 60-80 |
| Polyethylene glycol | Permeability enhancer | 40-20 |
| Coating concentration | — | 4-22% of |
| | | core tablet |
| | | weight |

TABLE A-2
Drug layer and sweller layer formulations
for BMS-986165 swellable core tablet

| | | Composition | |
|---|---|---|---|
| Ingredient | Function | % of blend | mg/tablet |

Drug Layer
Intragranular

| | | | |
|---|---|---|---|
| BMS-986165-01 SDD (15% | Active | 25.00 | 113.3 |
| BMS 986165-01:85% HPMCAS) | | | |
| PolyOx (Sentry PolyOx WSR | Entraining | 54.00 | 244.8 |
| N750 LEO) | polymer | | |
| HPMCAS-HF | Sustaining | 10.00 | 45.3 |
| | polymer | | |
| Sodium Chloride (powder) | Osmogen | 10.00 | 45.3 |
| Magnesium Stearate | Lubricant | 0.25 | 1.1 |

Extragranular

| | | | |
|---|---|---|---|
| Silicon Dioxide (Syloid 244 | Glidant | 0.50 | 2.3 |
| FP) | | | |
| Magnesium Stearate | Lubricant | 0.25 | 1.1 |

| | | |
|---|---|---|
| Drug Layer Total | 100.00 | 453.3 |

Sweller Layer

| | | | |
|---|---|---|---|
| PolyOx (PolyOx WSR | Entraining | 65.00 | 147.4 |
| Coagulant) | polymer | | |
| Microcrystalline Cellulose | Filler | 25.80 | 58.5 |
| (Avicel PH200) | | | |
| Sodium Chloride (powder) | Osmogen | 8.50 | 19.3 |
| Iron Oxide | Colorant | 0.20 | 0.4 |
| Magnesium Stearate | Lubricant | 0.50 | 1.1 |

| | | |
|---|---|---|
| Sweller Layer Total | 100.00 | 226.7 |
| Total core tablet weight (mg) | | 680.0 |
| Tablet dose (mg) | | 17 |

TABLE A-3
Coating compositions

| Coating composition | |
|---|---|
| (Cellulose acetate:polyethylene | |
| glycol) | Coating amount |
| 70:30 | 4-22 wt % |
| 80:20 | 4-22 wt % |

What is claimed is:

1. A swellable core dosage form comprising a dispersion of amorphous 6-(cyclopropaneamido)-4-((2-methoxy-3-(1-methyl-1H-1,2,4-triazol-3-yl)phenyl)amino)-N-(methyl-d₃)pyridazine-3-carboxamide (BMS-986165) dispersed in a polymer matrix.

2. A method of treating an autoimmune disease or auto-inflammatory disease in a subject, the method comprising administering to the subject the swellable core dosage form according to claim 1.

3. A method of treating an inflammatory bowel disease in a subject, the method comprising administering to the subject the swellable core dosage form according to claim 1.

4. The method according to claim 3, wherein the inflammatory bowel disease is ulcerative colitis.

5. The method according to claim 3, wherein the inflammatory bowel disease is Crohn's disease.

6. A method of treating psoriasis in a subject, the method comprising administering to the subject the swellable core dosage form according to claim 1.

7. The method according to claim 6, wherein the psoriasis is plaque psoriasis.

8. The method according to any one of claims 2-7, wherein the swellable core dosage form is a bi-layer tablet.
